(19) United States
(12) Patent Application Publication
(10) Pub. No.: US 2025/0262229 A1
(43) Pub. Date: Aug. 21, 2025

(54) APPLICATION OF PHD2 INHIBITOR IN PREPARATION OF DRUGS FOR PREVENTION AND TREATMENT OF PLATELET THROMBUS

(71) Applicant: Nankai University, Tianjin (CN)

(72) Inventors: Gang Bai, Tianjin (CN); Yuanyuan Hou, Tianjin (CN); Chuanjing Cheng, Tianjin (CN); Kaixin Liu, Tianjin (CN)

(21) Appl. No.: 18/950,182

(22) Filed: Nov. 18, 2024

(30) Foreign Application Priority Data
Feb. 19, 2024 (CN) 202410182534.4

(51) Int. Cl. A61K 31/7034; A61K 31/4704; A61K 31/515; A61K 31/704; A61P 7/02

(52) U.S. Cl. CPC A61K 31/7034; A61K 31/4704; A61K 31/515; A61K 31/704; A61P 7/02

(57) ABSTRACT

An application of a prolyl hydroxylase domain 2 (PHD2) in preparation of drugs for prevention and treatment of platelet thrombus is provided and relates to the field of biomedical technology. The important role of PHD2 protein in the process of platelet adhesion and thrombus formation is provided, PHD2 as a regulatory target of platelet adhesion is also provided, and thrombus formation can be significantly inhibited by knocking down PHD2 gene expression. This provides new technological ways for the development of drugs for antiplatelet thrombus and the improvement of blood microcirculation disorders. The PHD2 inhibitor can inhibit platelet thrombus by inhibiting PHD2, thereby providing new therapeutic options and candidate drugs for the prevention and/or treatment of platelet thrombus diseases due to platelet excessive activation (including, but not limited to arterial thrombosis, such as heart attack and stroke, and venous thrombus diseases, such as pulmonary embolism and deep vein thrombosis).

### CROSS-REFERENCE TO RELATED APPLICATION

This application claims priority to Chinese Patent Application No. 202410182534.4, filed Feb. 19, 2024, which is herein incorporated by reference in its entirety.

### TECHNICAL FIELD

The disclosure relates to the field of biomedical technologies, and more particularly to an application of a prolyl hydroxylase domain 2 (PHD2) inhibitor in preparation of a drug for prevention and/or treatment of platelet thrombus.

### STATEMENT REGARDING SEQUENCE LISTING

The sequence listing associated with this application is provided in text format in lieu of a paper copy and is hereby incorporated by reference into the specification. The name of the XML file containing the sequence listing is 24065THXT-USP1-SL.xml. The XML file is 1,786 bytes; is created on Oct. 30, 2024; and is being submitted electronically via patent center.

### BACKGROUND

The lesions caused or complicated by thromboembolism are about three times as many as those of carcinoma, which seriously jeopardize human health. Therefore, prevention of thrombosis is a focus and hotspot of contemporary medical research. Platelets play an important role in physiological hemostasis and pathological thrombosis. Under physiological conditions, platelets maintain the homeostasis of the intravascular environment and prevent massive blood loss after injury to the organism. After injury and bleeding, platelets participate in the normal hemostatic process and play a protective role for the organism. However, under pathological conditions, platelets are excessively activated in the blood, forming thrombi to block blood vessels, resulting in abnormal blood flow into tissues, thus causing ischemia in tissues and inducing ischemic diseases. Platelets play an important role in the process of pathological thrombosis. With the increasing trend of population aging year by year, a variety of ischemic diseases caused by thrombosis are the main cause of death in middle-aged and elderly people, which poses a serious threat to the health and quality of life. Therefore, platelet therapy has become one of the most important directions in the prevention and treatment of thrombotic diseases.

After injury to the vessel wall, collagen from the extracellular matrix binds to platelet receptors, triggering additional platelet adhesion and aggregation, while activating the coagulation cascade, followed by an enzymatic reaction to produce insoluble fibrin, which ultimately forms thrombi. Platelets are in a resting state in normal circulating blood. When blood vessels are damaged and expose structures such as subendothelial collagen, platelets will adhere to the subendothelial tissue at the site of vascular damage by directly binding to plasma proteins through their surface membrane glycoproteins, such as von Willebrand factor (VWF). Adherent platelets or platelets that are activated by platelet agonists produced during the coagulation process or tissue injury undergo a change in shape, extending pseudopods, and subsequently releasing contents of their intracellular granules. At the same time, the platelet membrane glycoprotein (GP) IIb-IIIa complex is activated to form an adhesion molecule receptor, which binds to fibrinogen, prompting mutual adhesion and aggregation of platelets, ultimately leading to the formation of a platelet thrombus at the site of vascular damage.

At present, platelet thrombus inhibitors mainly include inhibitors of platelet activation and aggregation, drugs that affect the coagulation cascade, and thrombolytic agents. Many of these drugs have a single route of administration and come with risks of bleeding, potential drug interactions, or resistance, which limit the clinical application of existing antiplatelet drugs.

Prolyl hydroxylase domain 2 (PHD2) is a 2-oxoglutarate-dependent dioxygenase that plays a crucial role in proline hydroxylation, contributing to the stabilization of protein structures. Since PHD2 is involved in regulating hypoxia-inducible factor 1 (HIF-1) signaling, PHD2 inhibitors have attracted widespread attention as potential therapeutic targets for various diseases, including anemia, ischemic heart disease, stroke, cancer, and pulmonary hypertension. Chinese patents have disclosed a series of patents related to PHD2, including application of prolyl hydroxylase inhibitors in prevention and treatment of iron deficiency and related diseases (CN115317589A); use of PHD inhibitors for protection, treatment, or alleviation of diseases related to HIF and/or erythropoietin (EPO) in patients (CN108069957B); and application of PHD inhibitors in preparation of drugs for prevention and treatment of acute kidney injury (CN108434139B), etc. Currently, there have been no reports on the use of PHD2 inhibitors to reduce platelet adhesion in the prevention and treatment of diseases related to antiplatelet thrombus.

### SUMMARY

An objective of the disclosure is to provide an application of a prolyl hydroxylase domain 2 (PHD2) inhibitor in preparation of drugs for prevention and/or treatment of platelet thrombus, to solve the problems of the above-mentioned related art. The disclosure discovers that PHD2 can serve as a regulatory target for platelet adhesion. The PHD2 inhibitors can inhibit the formation of platelet thrombus by inhibiting PHD2, thereby providing a new therapeutic scheme and candidate drugs for the prevention and/or treatment of platelet thrombotic diseases.

In order to achieve the above objective, the disclosure provides the following technical solutions.

The disclosure provides an application of a PHD2 inhibitor in preparation of drugs for prevention and/or treatment of platelet thrombus.

In an embodiment, the PHD2 inhibitor is at least one selected from the group consisting of Daprodustat, Roxadustat, Vadadustat, IOX2 (with formal name of N-[[1,2-dihydro-4-hydroxy-2-oxo-1-(phenylmethyl)-3-quinolinyl]carbonyl]-glycine, molecular formula of C₁₉H₁₆N₂O₅), ginsenoside CK, and polydatin.

The disclosure also provides a drug for prevention and/or treatment of platelet thrombus, an active ingredient of the drug includes a PHD2 inhibitor.

In an embodiment, the PHD2 inhibitor is at least one selected from the group consisting of Daprodustat, Roxadustat, Vadadustat, IOX2, ginsenoside CK, and polydatin.

The disclosure also provides an application of ginsenoside CK in preparation of a PHD2 inhibitor.

The disclosure also provides an application of polydatin in preparation of a PHD2 inhibitor.

The disclosure also provides an application of a PHD2 inhibitor in preparation of a drug for alleviating blood circulation disorder caused by acute soft tissue injury.

In an embodiment, the PHD2 inhibitor is at least one selected from the group consisting of Daprodustat, Roxadustat, Vadadustat, IOX2, ginsenoside CK, and polydatin.

The disclosure also provides an application of a PHD2 inhibitor in preparation of a drug for ameliorating dysfunction of blood coagulation induced by bacterial infection.

In an embodiment, the PHD2 inhibitor is at least one selected from the group consisting of Daprodustat, Roxadustat, Vadadustat, IOX2, ginsenoside CK, and polydatin.

The disclosure discloses the following technical effects.

The disclosure discloses the important role of PHD2 protein in the process of platelet adhesion and thrombus formation, provides PHD2 as a regulatory target of platelet adhesion. By knocking down the expression of the PHD2 gene, the thrombus formation can be significantly inhibited. This provides new technological means for the development of drugs for antiplatelet thrombus and the improvement of blood microcirculation disorders.

The disclosure finds that the PHD2 inhibitor can inhibit platelet thrombus by inhibiting PHD2, thereby providing new therapeutic options and candidate drugs for the prevention and/or treatment of platelet thrombus diseases due to platelet excessive activation (including, but not limited to arterial thrombosis, such as heart attack and stroke, and venous thrombus diseases, such as pulmonary embolism and deep vein thrombosis).

The disclosure also finds that the active natural products ginsenoside CK and polydatin can serve as the PHD2 inhibitor for reducing platelet coagulation and ameliorating platelet thrombus diseases. The ginsenoside CK has an ameliorating effect on blood circulation disorders and platelet thrombus triggered by acute tissue injury; and polydatin has an ameliorating effect on dysfunction of blood coagulation triggered by bacterial infection.

### BRIEF DESCRIPTION OF DRAWINGS

In order to illustrate technical solutions in embodiments or related art of the disclosure more clearly, the accompanying drawings to be used in the embodiments will be briefly introduced below. Apparently, the accompanying drawings in the following description are only some of the embodiments of the disclosure, and for those skilled in the related art, other accompanying drawings can be obtained based on these drawings without paying in creative labor.

FIG. 1 illustrates a schematic diagram of principle of preventing and treating platelet thrombus by a prolyl hydroxylase domain 2 (PHD2) inhibitor.

FIGS. 2A-2E illustrate effects of PHD2 gene expression on disseminated intravascular coagulation in mice. Specifically, FIG. 2A illustrates results of assessing the efficiency of PHD2 gene knockdown by western blotting analysis. FIG. 2B illustrates effects of PHD2 gene knockdown on tail bleeding volume. FIG. 2C illustrates effects of PHD2 gene knockdown on tail bleeding time. FIG. 2D illustrates results of pulmonary thrombi in mice with PHD2 gene knockdown by Masson staining analysis. FIG. 2E illustrates results of pulmonary thrombi in mice with PHD2 gene knockdown by Masson scoring statistics.

FIGS. 3A-3C illustrate effects of PHD2 inhibitors on platelet adhesion. Specifically, FIG. 3A illustrates chemical structural formulas of the four PHD2 inhibitors. FIG. 3B illustrates effects of the PHD2 inhibitors on PHD2 enzyme activity. FIG. 3C illustrates effects of the PHD2 inhibitors on platelet adhesion.

FIGS. 4A-4D illustrate effects of Daprodustat on lipopolysaccharide-induced disseminated intravascular coagulation in mice. Specifically, FIG. 4A illustrates detection results of the tail bleeding volume in respective groups. FIG. 4B illustrates detection results of the tail bleeding time in respective groups. FIG. 4C illustrates results of the pulmonary thrombi in respective groups by Masson staining analysis. FIG. 4D illustrates results of the pulmonary thrombi in respective groups by Masson scoring statistics.

FIGS. 5A-5D illustrate effects of IOX2 on disruption of platelet adhesion by binding of von Willebrand factor (VWF) to collagen. Specifically, FIG. 5A illustrates results of platelet adhesion by IOX2 by fluorescence imaging analysis. FIG. 5B illustrates results of collagen-platelet adhesion assay. FIG. 5C illustrates results of collagen-VWF binding assay. FIG. 5D illustrates results of collagen-VWF-platelet adhesion assay.

FIGS. 6A-6F illustrate results of interaction analysis of ginsenoside CK and polydatin with PHD2 respectively. Specifically, FIG. 6A illustrates a chemical structural formula of the ginsenoside CK. FIG. 6B illustrates results of binding constants between the ginsenoside CK and the PHD2 determined by surface plasmon resonance (SPR) technique. FIG. 6C illustrates effects of the ginsenoside CK on the PHD2 enzyme activity. FIG. 6D illustrates a chemical structural formula of the polydatin. FIG. 6E illustrates results of binding constants between the polydatin and the PHD2 determined by the SPR technique. FIG. 6F illustrates effects of the polydatin on the PHD2 enzyme activity.

FIGS. 7A-7H illustrate effects of the ginsenoside CK on blood microcirculation disorders induced by soft tissue injury in rats. Specifically, FIGS. 7A-7D illustrate results of whole blood viscosity assay at 1 second(s), 3 s, 30 s and 200 s in respective groups, respectively. FIG. 7E illustrates results of erythrocyte aggregation index assay in respective groups. FIG. 7F illustrates results of erythrocyte packed-cell volume assay in respective groups. FIG. 7G illustrates results of platelet number assay in respective groups. FIG. 7H illustrates results of mean platelet volume assay in respective groups.

FIGS. 8A-8D illustrate effects of the polydatin on improvement of in vivo coagulation indicators in mice induced by Pseudomonas aeruginosa. Specifically, FIG. 8A illustrates detection results of prothrombin time in respective groups. FIG. 8B illustrates detection results of activated partial thrombin time in respective groups. FIG. 8C illustrates detection results of thrombin time in respective groups. FIG. 8D illustrates detection results of fibrinogen content in respective groups.

### DETAILED DESCRIPTION OF EMBODIMENTS

The following is a detailed description of various exemplary embodiments of the disclosure. This detailed description should not be construed as a limitation on the disclosure, but should be understood as a more detailed description of certain aspects, characteristics and embodiments of the disclosure.

It should be understood that terminologies described in the disclosure are merely for describing specific embodiments and are not intended to limit the disclosure. In addition, for numerical ranges in the disclosure, it should be understood that each intermediate value between the upper limit and the lower limit of the range is also specifically disclosed. Intermediate values within any stated value or stated range, and each smaller range between any other stated value or intermediate values within the stated range are also included in the disclosure. The upper and lower limits of these smaller ranges can be independently included or excluded from the range.

Unless otherwise specified, all technical and scientific terminologies used herein have the same meaning as commonly understood by those skilled in the art to which this disclosure pertains. Although the disclosure only describes the illustrated methods and materials, any methods and materials similar or equivalent to those described herein can also be used in the implementation or testing of the disclosure. All documents mentioned in this specification are incorporated by reference to disclose and describe methods and/or materials related to the documents. In case of conflict with any incorporated document, the contents of this specification shall prevail.

Without departing from the scope or spirit of the disclosure, various improvements and modifications made to the specific embodiments described in the specification will be apparent to those skilled in the art. Other embodiments will be apparent to those skilled in the art from the description of the disclosure. The description and embodiments of the disclosure are exemplary only.

The terms “comprise”, “include”, “have” and “contain” used in the disclosure are all open-ended terms, meaning that they include but are not limited to.

Daprodustat (Cat. No. HY-17608), Roxadustat (Cat. No. HY-13426), Vadadustat (Cat. No. HY-101277), and IOX2 (Cat. No. HY-15468) used in the following embodiments are purchased from MedChemExpress (MCE®). Ginsenoside CK (also referred to as compound K with molecular formula of C₃₆H₆₂O₈, Cat. No. S29921) and polydatin (with molecular formula of C₂₀H₂₂O₈, Cat. No. B20533) are purchased from Shanghai yuanye Bio-Technology Co., Ltd. The experimental animals used are male ICR (CD-1) mice weighing 18-22 grams (g) and Sprague-Dawley (SD) rats weighing 200-220 g, purchased from Beijing Vital River Laboratory Animal Technology Co., Ltd., China.

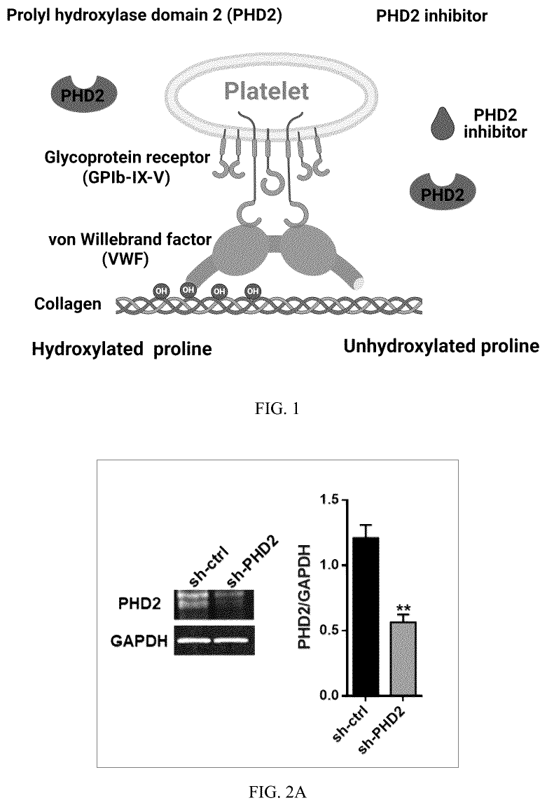

The disclosure finds that prolyl hydroxylase domain 2 (PHD2) inhibitors reduce prolyl hydroxylation of collagen by inhibiting PHD2 enzyme activity, prevents adhesion of von Willebrand factor (VWF) to collagen, and thus slowing down platelet adhesion function. The principle is shown in FIG. 1 and is described in detail as follows.

### Embodiment 1 Effect of Knockdown of PHD2 Gene on Mouse Disseminated Intravascular Coagulation Model

The short hairpin RNA against PHD2 (shPHD2) plasmid (target sequence: 5′-AGACTGGGGACGCCAAGGTA-3′ as shown in SEQ ID NO: 1) is designed and synthesized by WZ Bioscience Inc in Shandong, China. The PHD2 gene Is knocked down using the adeno-associated virus serotype 9 (AAV9) vector pAV-U6-shRNA-CMV-Intron-mCherry with RNA interference technology.

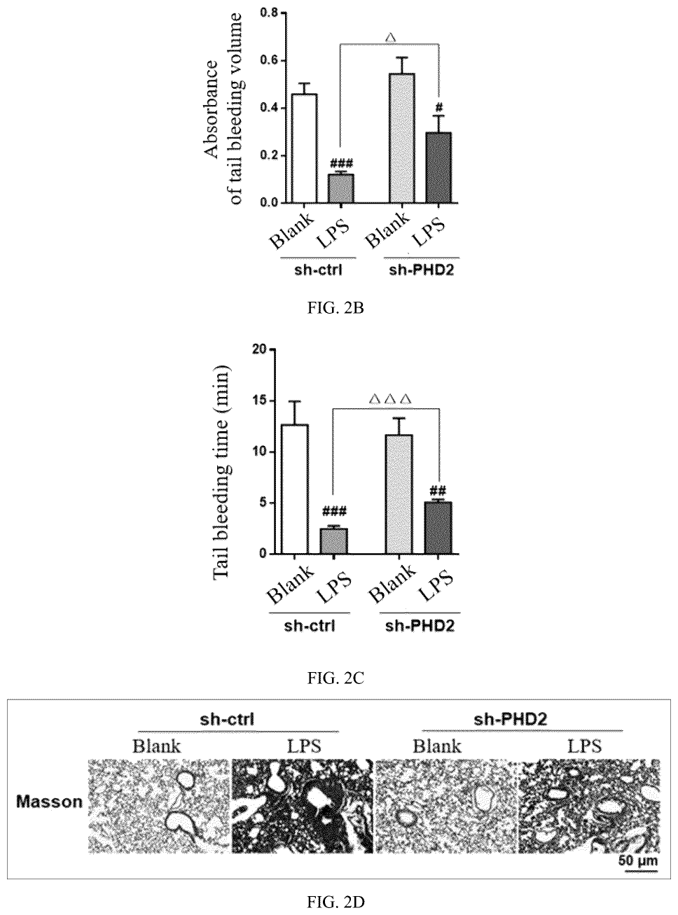

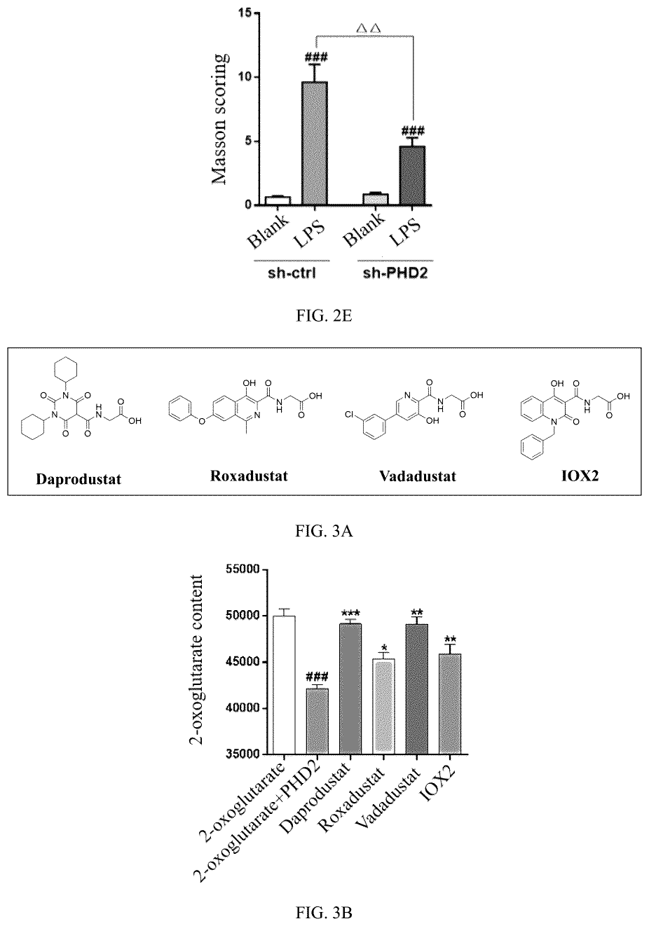

A PHD2 gene knockdown animal model is constructed as follows. 20 microliters (μL) (2.5×10¹³ vector genomes per milliliter, abbreviated as vg/mL) of shPHD2-expressing AAV9 virus (sh-PHD2 group) or negative control (sh-ctrl) virus is titrated into the trachea of mice and maintained for 2 weeks. The expression efficiency of the PHD2 gene in the lungs is assessed using western blotting. The results are shown in FIG. 2A. Compared with the negative control group, the PHD2 protein expression in the sh-PHD2 group is significantly reduced, and the knockdown efficiency is about 60%, indicating that the PHD2 gene knockdown animal model is successfully constructed.

The sh-PHD2 mice and sh-ctrl blank mice are randomly divided into a blank group and a model group, with 6 mice in each group. The model group is injected intraperitoneally with 10 milligrams per kilogram (mg/kg) lipopolysaccharide abbreviated as LPS (Beijing Solarbio Science & Technology Co., Ltd., Cat. No. L8880) to induce the mice to establish a disseminated intravascular coagulation (DIC) model, and the blank group is injected intraperitoneally with the corresponding volume of physiological saline. After 2 hours, the mice are anesthetized by intraperitoneal injection of 1% pentobarbital sodium (40 mg/kg), and the tail is cut at a site of 3 millimeters (mm) from the tip of the tail of each mouse, and the tail is placed vertically in 37° C. physiological saline immediately after the first drop of blood is sucked up on filter paper to record the tail bleeding time. The results are shown in FIG. 2B and FIG. 2C, show that LPS-induced DIC in sh-ctrl blank mice results in a significant decrease in the tail bleeding time and the tail bleeding volume, whereas in sh-PHD2 mice, the tail bleeding time and the tail bleeding volume in the model group are significantly increased compared with those in the blank group. The results of Masson staining of paraffin sections of lung tissues fixed in 10% formalin in respective groups are shown in FIG. 2D. The results show that the blank group exhibits bleeding induced by LPS, along with extensive accumulation of collagen fibers and multiple thrombi in pulmonary blood vessels; in contrast, the above phenomena in the sh-PHD2 mice are significantly ameliorated.

In summary, the results indicate that PHD2 gene knockdown has a significant effect on coagulation function, suggesting that PHD2 inhibitors have a positive effect on the prevention and treatment of platelet thrombus (compared with the sh-ctrl blank group, **P<0.01; compared with the LPS group, ^{Δ}P<0.05; ^{ΔΔ}P<0.01; ^{ΔΔΔ}P<0.001; compared with the blank group, ^{#}P<0.05; ^{##}P<0.01; ^{###}P<0.001; n=6).

### Embodiment 2 Effect of PHD2 Inhibitors on Platelet Adhesion

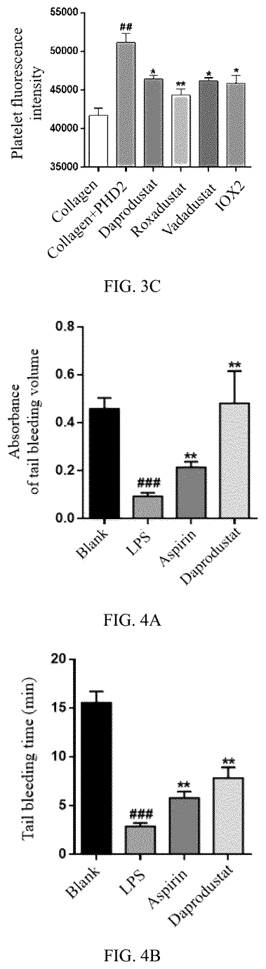

Four kinds of PHD2 inhibitors are selected, among which Daprodustat, Roxadustat, and Vadadustat are clinical drugs for the treatment of renal anemia, IOX2 is an inhibitor of chemical synthesis of PHD2, and their chemical structural formulas are shown in FIG. 3A.

1 micromole per liter (μM) PHD2 inhibitor is incubated with 100 micrograms per milliliter (μg/mL) PHD2 protein (Abcam, Cat. No. ab132273) at 4° C. for 8 hours, and its inhibitory effect on PHD2 enzyme activity is determined according to the 2-oxogalarate-phenylenediamine method. The determination method is as follows.

100 μL of PHD2 enzyme solution incubated with the PHD2 inhibitor is taken and 100 μL of reaction substrate (300 μM of 2-oxogalarate, 150 μM of Fe²⁺) is added, followed by 100 μL of 0.5 moles (M) of hydrogen chloride (HCl) and 10 mg/mL of o-phenylenediamine, and the reaction is carried out at 95° C. for 15 minutes after mixing, so as to obtain a reaction product. The reaction product is centrifuged to taken the supernatant, 10 μL of 1.25 M of sodium hydroxide (NaOH) is added to terminate the reaction, and the fluorescence value of the reaction is determined using 340 nanometers (nm) excitation at the emission wavelength of 420 nm, the results are shown in FIG. 3B. The results show that the four PHD2 inhibitors have certain inhibitory effect on the enzyme activity, among which Daprodustat has the optimal effect (compared with the blank control group of 2-oxogalarate, ^{###}P<0.001; compared with the 2-oxogalarate+PHD2 group, *P<0.05, **P<0.01, ***P<0.001; n=6.

In addition, 10 μg/mL type I collagen (Beijing Solarbio Science & Technology Co., Ltd., Cat. No. C8062) is mixed with the above PHD2 enzyme solution after reaction with the PHD2 inhibitor, and added to a black 96-well microplate and incubated at 4° C. overnight. SD rat platelets (2×10⁷ cells/mL) are then inoculated on the microplate and incubated at 37° C. for 90 minutes. After washing with phosphate-buffered saline (PBS), platelets are stained with Calcein-AM dye (Shanghai yuanye Bio-Technology Co., Ltd., Cat. No. S25004), and fluorescence values of the platelets are measured with a multifunctional microplate reader under conditions of 488 nm excitation and 525 nm emission wavelength detection to investigate platelet adhesion, the results are shown in FIG. 3C. The results show that the above PHD2 inhibitors can inhibit platelet adhesion to collagen, and there is no significant difference in the inhibitory effect (compared with the collagen group, ^{##}P<0.01; compared with the collagen +PHD2 group, *P<0.05, **P<0.01; n=6).

### Embodiment 3 Effect of Daprodustat on LPS-Induced DIC in Mice

ICR (CD-1) mice are randomly divided into blank group, LPS model group, aspirin group (10 mg/kg, taken orally, purchased from Beijing Solarbio Science & Technology Co., Ltd., Cat. No. A8830) and Daprodustat group (intraperitoneal injection, 10 mg/kg), with 6 mice in each group. In addition to the blank group, the other groups use the method of the embodiment 1 to prepare the LPS-induced DIC model in mice, and the administration group is given drug intervention 1 hour before modeling.

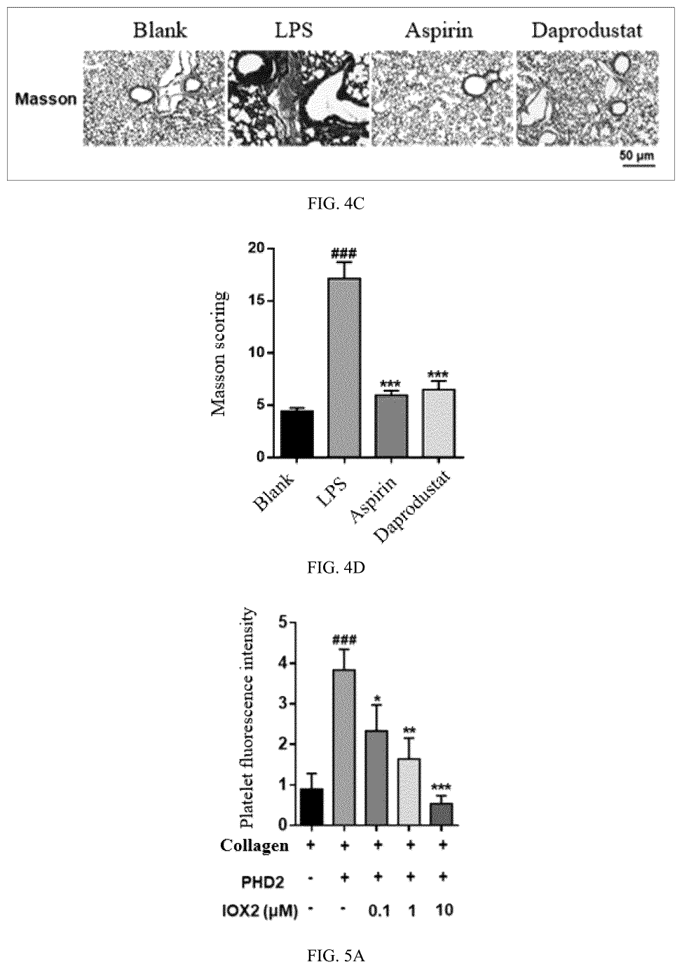

One hour after oral administration of aspirin, half an hour after intraperitoneal injection of Daprodustat, the intravascular coagulation is investigated, and the tail bleeding volume and bleeding time of mice are recorded. The results are shown in FIG. 4A and FIG. 4B, the bleeding time of mice after DIC induced by LPS is decreased significantly, while oral aspirin or intraperitoneal injection of Daprodustat can significantly increase the tail bleeding time and the tail bleeding volume of model mice. Masson staining results of lung tissues of respective groups (as shown in FIG. 4C) also show that bleeding and collagen fiber accumulation occurred after LPS modeling, and multiple thrombi are formed in the lungs. In contrast, the PHD2 inhibitor Daprodustat or the antithrombotic inhibitor aspirin can significantly improve the above phenomenon and significantly inhibit the formation of thrombus (compared with the blank group, ^{##}P<0.01; compared with the LPS group, **P<0.01; ***P<0.001; n=6).

### Embodiment 4 IOX2 Affects the Binding of VWF to Collagen and Destroys Platelet Adhesion

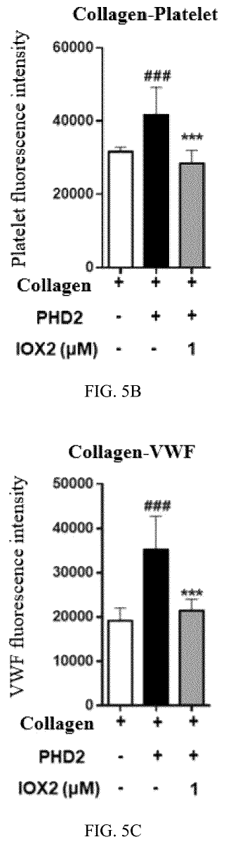

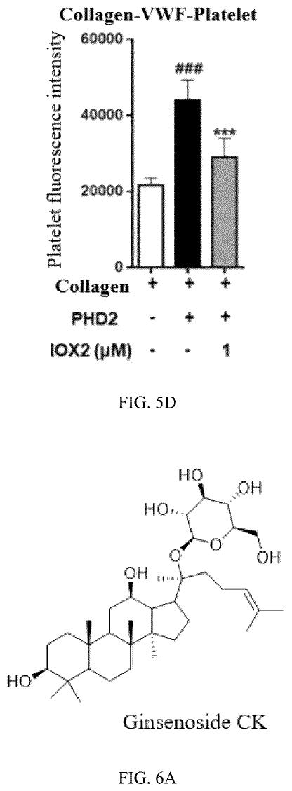

Different doses of PHD2 inhibitor IOX2 (0.1 μM, 1 μM, 10 μM) are taken and incubated with 100 μg/mL PHD2 protein at 4° C. for 8 hours, then 10 μg/mL type I collagen is mixed with the above solution and added dropwise to glass slides, and incubated at 4° C. overnight. SD rat platelets (2×10⁷ cells/mL) are then inoculated on the glass slides and incubated at 37° C. for 90 minutes. After being fully washed by PBS, the fixed platelets are stained with Calcein-AM dye, fluorescence imaging analysis is performed using fluorescence microscope (Leica TCS SP8, Germany), and statistical analysis is performed on platelets with collagen adhesion using Image J software. The results are shown as in FIG. 5A. IOX2 inhibits platelet adhesion to collagen in a concentration-dependent manner, significantly reducing the occurrence of platelet adhesion, indicating that the PHD2 inhibitor has a significant effect on platelet adhesion.

The binding of platelets to collagen occurs mainly through VWF as a junction. PHD2 promotes the hydroxylation of collagen to form a three-dimensional structure to better bind to VWF. To verify the above principle, 10 μg/mL type I collagen is mixed with 1 μM IOX2-treated PHD2 protein and inoculated into a black 96-well plate, incubated at 4° C. overnight. Parallel triplicates, for the first one, SD rat platelets (2×10⁷ cells/mL) are added and incubated at 37° C. for 90 minutes, after full washing with PBS, the fixed platelets are stained with Calcein-AM dye. For the second one, the primary antibody of VWF (Beijing Boaosen Biology technology Co., LTD., bs-20428R) is incubated at 4° C. overnight, after the excess primary antibody is washed away by PBS, the fluorescent secondary antibody (Abcam, ab150077) is incubated at 37° C. for 1 hour, and the excess secondary antibody is washed away. For the third one, SD rat plasma is added, incubated at room temperature for 4 hours, and then SD rat platelets (2×10⁷ cells/mL) are added, incubated at 37° C. for 90 minutes, after full washing with PBS, the fixed platelets are stained with Calcein-AM dye. The fluorescence values of respective groups are measured by a multifunctional microplate reader at the excitation wavelength of 488 nm and the emission wavelength of 525 nm. The results are shown in FIG. 5B, indicating that IOX2 affects PHD2-mediated collagen-platelet adhesion by affecting the binding of collagen-VWF (FIG. 5C), which results in blocking the binding of collagen-VWF-platelet adhesion (FIG. 5D). This embodiment demonstrates that the PHD2 inhibitor IOX2 significantly reduces the occurrence of PHD2-mediated platelet adhesion (compared to the collagen group, ^{###}P<0.001; compared with the collagen+PHD2 group, *P<0.05, **P<0.01, ***P<0.001; n=3).

### Embodiment 5 Interaction Analysis of Active Natural Products with PHD2 Protein

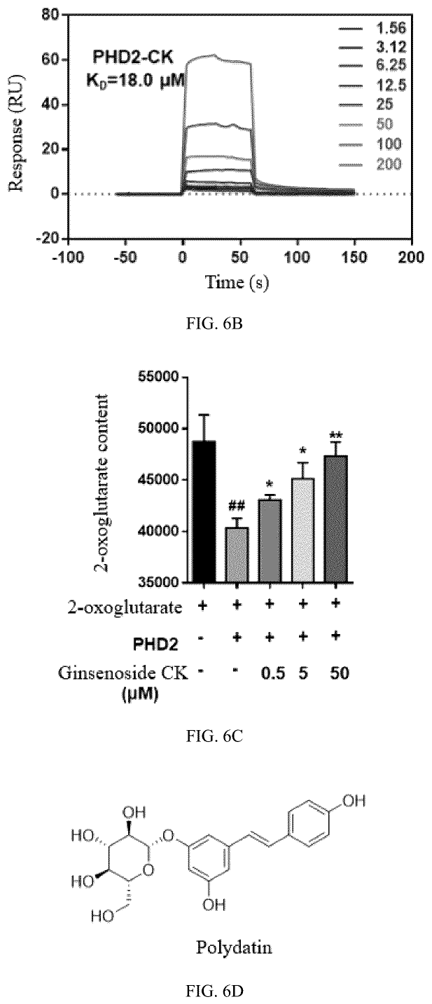

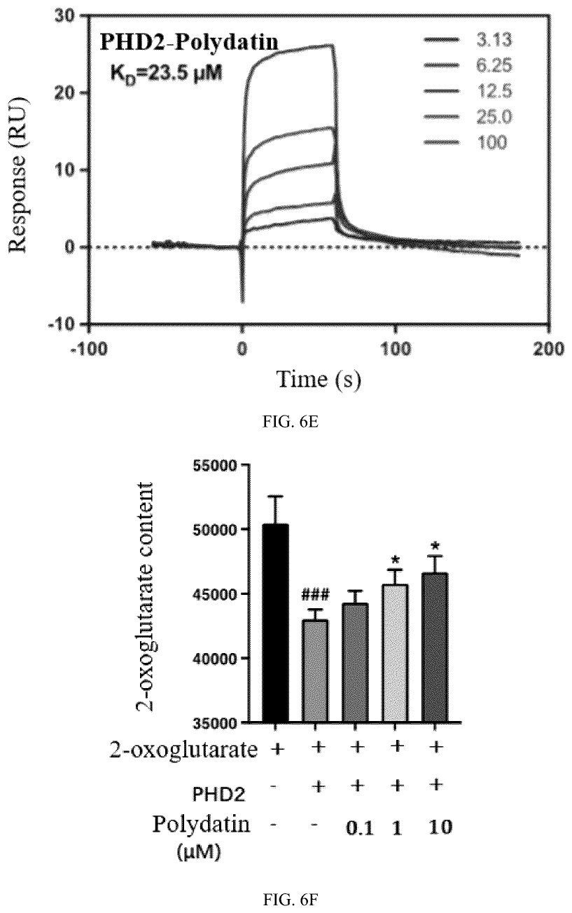

The chemical structure formulas of ginsenosides CK and polydatin are shown in FIG. 6A and FIG. 6D. The interaction between these active natural products and recombinant human PHD2 protein is analyzed by surface plasmon resonance (SPR) technique using a Biacore™ T200 surface plasmon resonance analyzer from GE healthcare. The CM5 chip is selected, the purified PHD2 protein is immobilized on the channel of the chip, and the active natural product solution as a mobile phase is flowed through the chip, with the concentration ranging from 1.56 μM to 200 μM. The flow rate of mobile phase is 30 μL/min, and the dissociation is monitored for 150 seconds. The dissociation constants K_{D} values of the ginsenoside CK and the polydatin with PHD2 protein are 18.0 μM and 23.5 μM, respectively (FIG. 6B and FIG. 6E).

In addition, the inhibitory effects of ginsenoside CK and polydatin on PHD2 enzyme activity are respectively determined according to the PHD2 enzyme activity determination method in the embodiment 2, and the results are shown in FIG. 6C and FIG. 6F. The results show that the ginsenoside CK at 0.5 μM, 5 μM, 50 μM and the polydatin at 0.1 μM, 1 μM, 10 μM can inhibit the PHD2 enzyme activity in a concentration gradient. (compared with the 2-oxoglutarate group, ^{###}P<0.001; compared with the 2-oxoglutarate+PHD2 group, *P<0.05, **P<0.01; n=3). These results suggest that the ginsenosides CK and the polydatin can serve as natural PHD2 inhibitors to reduce platelet aggregation and improve platelet thrombotic diseases.

### Embodiment 6 Effect of Ginsenoside CK on Blood Microcirculation Induced by Soft Tissue Injury in Rats

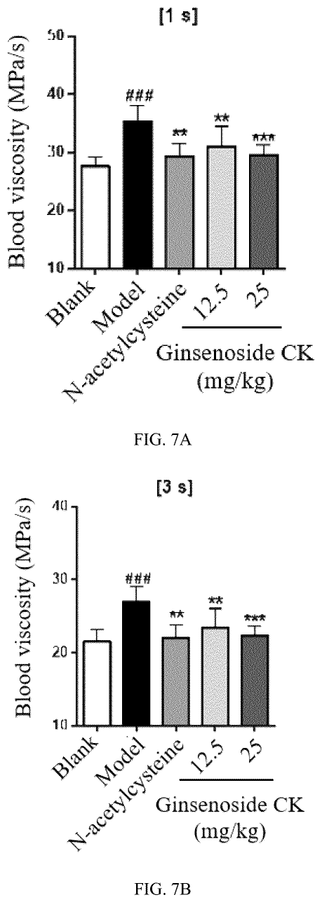

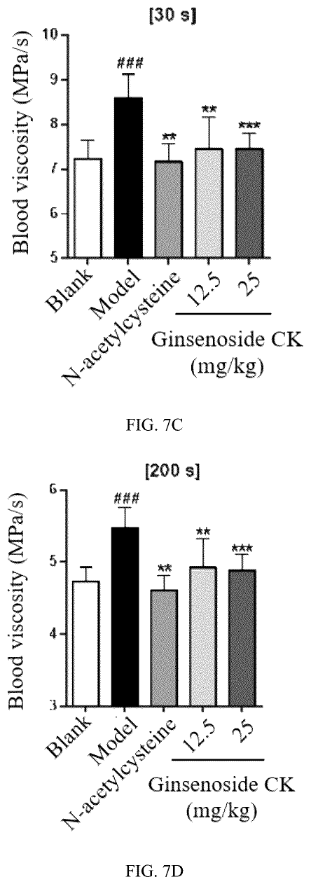

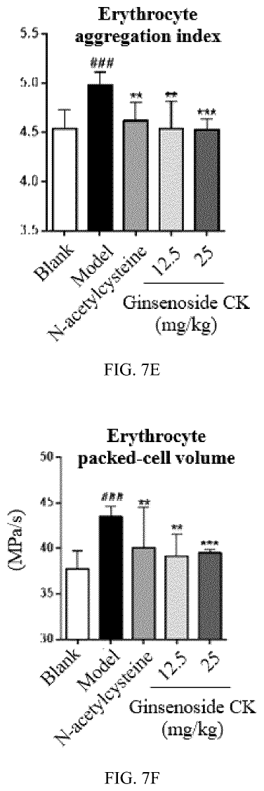

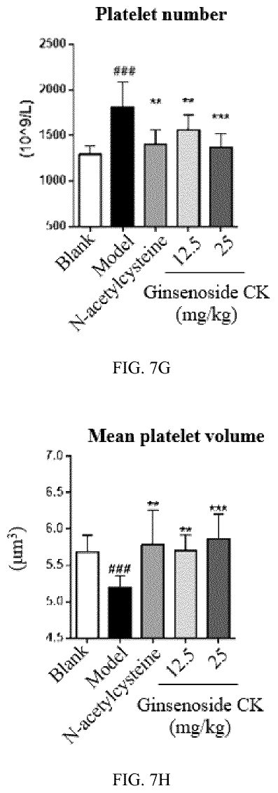

SD male rats are taken and randomly divided into a blank control group, a model group, a positive control group (N-acetylcysteine, intraperitoneally injected at 70 milligrams per kilogram per day abbreviated as mg/kg/d), and high and low dose ginsenoside CK groups (intraperitoneally injected at 25 mg/kg/d and 12.5 mg/kg/d). The blank control group and the model group are given the same volume of sterile drinking water in the same manner. The molding is investigated three days after pre-administration. The rats are first anesthetized with 1% pentobarbital sodium (40 mg/kg), and the hair of the right buttocks and posterior thighs of the anesthetized rats are removed and fixed on the plate of an acute soft tissue injury modeling device. A 100 g weight is selected to impact the lateral aspect of the mid-right calf with five free-falls from a height of 100 centimeters (cm) causing soft tissue injury. The rats with successful modeling are continued to be administered for 4 days, and 1 hour after the last administration, the rats are anesthetized, blood is collected from the abdominal aorta with a blood viscometer to detect the viscosity of whole blood of the rats with high shear rate [200 s], medium shear rate [30 s], low shear rate [3 s], and low shear rate [1 s]. Coagulation parameters, such as erythrocyte aggregation index, erythrocyte packed-cell volume, platelet number and mean platelet volume, are determined by using a blood cell analyzer. The results are shown in FIGS. 7A-7H.

As shown in FIGS. 7A-7H, compared with the blank control group, the whole blood viscosity of the model group is significantly increased at low shear rate, medium shear rate and high shear rate. Compared with the model group, the whole blood viscosity of the N-acetylcysteine group and ginsenoside CK treatment groups at different doses are improved to a certain extent. In addition, compared with the blank control group, the erythrocyte aggregation index, the erythrocyte packed-cell volume, and the platelet number in the model group are significantly increased, indicating increased blood viscosity and poor blood flow, thus promoting the formation of thrombus. The positive control group and the ginsenoside CK group can significantly reduce these indicators after administration of the intervention, and the mean platelet volume is also improved. These results indicate that the ginsenoside CK, a natural PHD2 inhibitor, has an ameliorating effect on blood circulation disorder and platelet thrombus formation induced by tissue injury (compared with the blank control group, ^{###}P<0.001; compared with the model group, **P<0.01, ***P<0.001; n=6).

### Embodiment 7 Effect of Polydatin on Ameliorating Blood Coagulation Function in Mice Induced by Pseudomonas aeruginosa

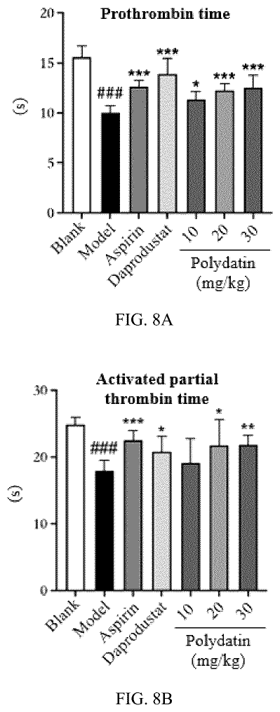

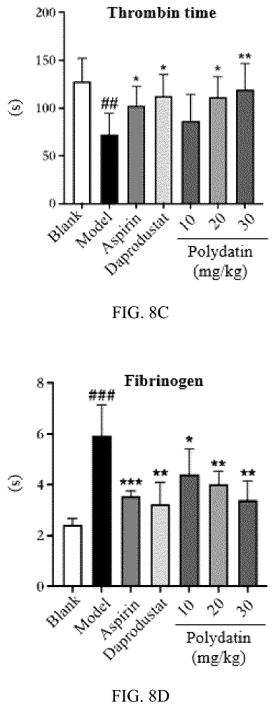

ICR (CD-1) male mice are taken and randomly divided into 7 groups (6 mice in each group), which are a blank control group, a model group, an aspirin positive control group (gavage at 30 mg/kg), a Daprodustat-positive control group (intraperitoneally injected at 15 mg/kg), and low, medium and high doses of polydatin group (intraperitoneally injected at 10 mg/kg, 20 mg/kg, 40 mg/kg). Mice are anesthetized by intraperitoneal injection of 1% pentobarbital sodium (40 mg/kg). Activated Pseudomonas aeruginosa (1×10⁷/20 μL) is added dropwise into the nasal cavity of mice in the model group and each administration group, and an equal amount of physiological saline is dropped into the blank control group. The respective groups are administered at 0 and 8 hours after molding, respectively. 24 hours after infection, plasma is obtained by blood sampling from mouse eyeballs, and four indexes of coagulation (prothrombin time, activated partial thrombin time, thrombin time and fibrinogen) are measured by a semi-automatic coagulation analyzer. The results are shown in FIGS. 8A-8D

As shown in FIGS. 8A-8D, compared with the blank control group, the prothrombin time, activated partial thrombin time and thrombin time of the model group are significantly shortened, and the fibrinogen content is significantly increased, indicating dysfunction of blood coagulation in vivo. After the administration of the intervention, the positive control group and the polydatin group can significantly prolong the prothrombin time, activated partial thrombin time and thrombin time of mice, and the fibrinogen content in blood can be reduced, which has a significant anticoagulant effect. The results show that the polydatin, a natural PHD2 inhibitor, has an ameliorating effect on dysfunction of blood coagulation induced by Pseudomonas aeruginosa infection (compared with the blank control group, ^{##}P<0.01, ^{###}P<0.001; compared with the model group, *P<0.05, **P<0.01, ***P<0.001; n=6).

The above embodiments only describe the illustrated implementation of the disclosure, and do not limit the scope of the disclosure. On the premise of not deviating from the design spirit of the disclosure, various modifications and improvements made by those skilled in the art to the technical solutions of the disclosure shall fall within the scope of protection defined in the claims of the disclosure.

What is claimed is:

1. An application method of a prolyl hydroxylase domain 2 (PHD2) inhibitor, comprising:
preparing a drug using the PHD2 inhibitor to prevent and treat platelet thrombus.

2. The application method as claimed in claim 1, wherein the PHD2 inhibitor is at least one selected from the group consisting of Daprodustat, Roxadustat, Vadadustat, IOX2, ginsenoside CK, and polydatin.

3. The application method as claimed in claim 1, comprising:
preparing the drug using ginsenoside CK as the PHD2 inhibitor to prevent and treat the platelet thrombus.

4. The application method as claimed in claim 1, comprising:
preparing the drug using polydatin as the PHD2 inhibitor to prevent and treat the platelet thrombus.

5. A drug for prevention and treatment of platelet thrombus, wherein an active ingredient of the drug comprises a PHD2 inhibitor.

6. The drug as claimed in claim 5, wherein the PHD2 inhibitor is at least one selected from the group consisting of Daprodustat, Roxadustat, Vadadustat, IOX2, ginsenoside CK, and polydatin.

7. An application method of a PHD2 inhibitor, comprising:
preparing a drug using the PHD2 inhibitor to alleviate blood circulation disorder caused by acute soft tissue injury.

8. The application method as claimed in claim 7, wherein the PHD2 inhibitor is at least one selected from the group consisting of Daprodustat, Roxadustat, Vadadustat, IOX2, ginsenoside CK, and polydatin.

9. The application method as claimed in claim 7, further comprising:
preparing a drug using the PHD2 inhibitor to ameliorate dysfunction of blood coagulation induced by bacterial infection.

10. The application method as claimed in claim 9, wherein the PHD2 inhibitor is at least one selected from the group consisting of Daprodustat, Roxadustat, Vadadustat, IOX2, ginsenoside CK, and polydatin.


SEQUENCE LISTING (1 sequence)

SEQ ID NO: 1 - DNA, 20 bp, synthetic construct
  source 1..20: mol_type = other DNA
agactggggacgccaaggta